(19) United States
(12) Patent Application Publication
(10) Pub. No.: US 2006/0185671 A1
(43) Pub. Date: Aug. 24, 2006

(54) Powder conveying pump

(75) Inventors: Thomas Durr, Tamm (DE); Hans-Georg Fritz, Ostfildern (DE); Frank Herre, Oberriexingen (DE)

Correspondence Address: YOUNG & BASILE, P.C., 3001 WEST BIG BEAVER ROAD, SUITE 624, TROY, MI, 48084, US

(73) Assignee: Durr Systems, Inc., Auburn Hills, MI (US)

(21) Appl. No.: 11/348,169

(22) Filed: Feb. 6, 2006

Related U.S. Application Data
(60) Provisional application No. 60/653,725, filed on Feb. 17, 2005.

(51) Int. Cl. A61M 15/00; A61M 11/00

(52) U.S. Cl. 128/203.15; 128/200.14

(57) ABSTRACT

A powder conveying pump, in particular for a powder coating device. The pump comprises a working chamber with a variable working chamber volume, a powder inlet opening into the working chamber to suck powder into the working chamber, a powder outlet opening out of the working chamber to expel the powder present in the working chamber and a moveable piston that forms one boundary surface of the working chamber and sucks the powder into the working chamber through the powder inlet during an upward movement. The piston is driven directly by pneumatic means.

### CROSS-REFERENCE TO RELATED APPLICATIONS

This application claims priority to U.S. Provisional Patent Application Ser. No. 60/653,725 filed on Feb. 17, 2005, the entire contents of which is incorporated herein in its entirety.

### FIELD OF THE INVENTION

The invention relates to a powder conveying pump, in particular for use in a powder coating installation.

### DESCRIPTION OF THE RELATED ART

A powder conveying pump is known from DE 101 45 448 A1 and from WO 03/024613 A1, which pump can be used in a station for coating media to convey the powder serving as the coating means. The known powder conveying pump has a piston driven mechanically through a piston rod, which generates a vacuum during an upward stroke in a working chamber of the powder conveying pump, sucking the powder into the working chamber of the powder conveying pump through a powder inlet. Transfer air is then blown into the working chamber, whereby the powder present in the working chamber is discharged through a powder outlet.

A disadvantage of this known powder conveying pump is the complex mechanical drive, which is realized by pneumatic cylinders through a piston rod.

### SUMMARY OF THE INVENTION

An object of the invention is, therefore, to simplify the drive in the case of the previously described known powder conveying pump. The invention contemplates driving the cylinder directly by pneumatic means so that a piston rod to connect the piston mechanically to the drive unit (e.g., pneumatic cylinder) can be eliminated. An additional advantage of the drive in accordance with the invention is that there are fewer moving parts and their mass is less, thereby making them less prone to wear and decreasing vibration.

A control chamber is preferably provided for the pneumatic piston drive, in which the piston forms one boundary surface of the control chamber. The pressure in the control chamber can be adjusted to apply appropriate pressure on the piston and to move same accordingly.

In one embodiment of the invention, the control chamber and the working chamber of the powder conveying pump are located on opposite sides of the piston, with the piston separating the control chamber from the working chamber. An increase in pressure in the control chamber effects a downward movement of the piston and a corresponding reduction in the volume of the working chamber, whereas a reduction of pressure in the control chamber effects an upward movement of the piston and results in a corresponding expansion of the volume in the working chamber. Thus, an upward movement does not necessarily specify a direction, it means movement away from the working chamber. Similarly, a downward movement is movement toward the working chamber.

In another embodiment of the invention, the control chamber and the working chamber are located on the same side of the piston so that the control chamber and the working chamber preferably act on the same face of the piston. For example, the working chamber can surround the control chamber in an annular configuration, with the control chamber separated from the working chamber by a cylindrical wall. The piston in this case has two axially-projecting and coaxially-positioned sub-pistons with an annular groove running between them. The cylindrical wall between the working chamber and the control chamber engages the annular groove. The disposition of the working chamber on the outside of the control chamber is advantageous because a greater working chamber volume is created thereby, resulting in a correspondingly higher delivery rate. An alternative possibility with this embodiment is that the control chamber surrounds the working chamber in an annular configuration.

Furthermore, the possibility exists with this embodiment that a control chamber is located on both sides of the piston to provide direct pneumatic drive to the piston, in which case the two control chambers act on opposite faces of the piston.

A positive pressure connection can open into the control chamber(s) for pneumatic drive to generate positive pressure in the control chamber and to move the piston accordingly, thereby changing the working chamber volume accordingly. If the control chamber is located on the side of the piston opposite the working chamber, an increase in pressure in the control chamber results in a downward movement of the piston and a corresponding decrease in working chamber volume. If, on the other hand, the control chamber is on the same side as the working chamber, an increase in pressure in the control chamber through the positive pressure connection results in an upward movement of the piston and a corresponding expansion of the working chamber volume.

Furthermore, a negative pressure connection can open into the control chamber(s) to reduce pressure in the control chamber, which results in a corresponding movement of the piston and a change in the working chamber volume.

Within the scope of the invention, pneumatic control of the piston can be exercised solely by generating positive pressure on both sides of the piston. However, an alternative possibility is to exercise pneumatic control of the piston solely by generating negative pressure on opposite sides of the piston. Furthermore, the possibility also exists of combining the generation of negative pressure and positive pressure, where negative pressure and positive pressure are preferably generated on the same side of the piston to move the piston correspondingly.

In the preferred embodiment of the invention, the movement of the piston serves only to suck powder into the working chamber, whereas the discharge of the powder from the working chamber through the powder outlet is not carried out by the piston. For this purpose, a transfer air connection is preferably provided that opens into the working chamber to blow transfer air into the working chamber and to discharge the powder previously sucked in and present there through the powder outlet.

The transport air connection and the powder outlet are preferably located opposite each other in the working chamber. This offers the advantage that the airflow pattern from the transport air connection to the powder outlet assists the discharge of the powder.

In addition, a negative pressure connection can also open into the working chamber to move the piston out of the working chamber after the powder has been expelled and to change the working chamber volume accordingly. After the powder has been discharged, the piston must first move down again (i.e., toward the working chamber) in order to reduce the volume of the working chamber so that powder can then be sucked into the working chamber once again.

The upward and downward movement of the piston can also be assisted or effected by a spring, where the spring can optionally push or pull the piston. The piston can be connected to a compression spring or a tension spring that moves the piston back to a starting position if there is no acting external pneumatic pressure. For example, the spring can move the piston upward (i.e., toward the control chamber) to suck in the powder, whereas the subsequent downward movement of the piston is driven solely by pneumatic means.

Furthermore, it is advantageous if at least one stop is located in the working chamber and/or in the control chamber, which stop restricts the movement of the piston in order to achieve a defined swept volume and thus produce a specified rate of delivery.

It is further advantageous if the piston consists of a elastic material in order to achieve the best possible sealing between the piston and the surrounding running surface. For example, the piston can consist of a plastic material that rides flexibly against the running surface. This is also advantageous because plastic is relatively light so that lower inertial forces occur during the oscillating operation of the piston, which reduces vibration.

The piston can further have a single-piece sealing lip formed on its outer surface to scrape powder remnants from the running surface without a separate piston ring. The piston can thus be constructed like a go-devil, which is used in modem coating plants to clean paint lines.

It should further be mentioned that the invention is not restricted to the previously described powder conveying pump in accordance with the invention, but includes a complete powder coating installation having such a powder conveying pump.

Furthermore, the invention includes the innovative use of such a powder conveying pump to transport powder in a powder coating installation.

Other advantageous refinements of the invention are identified in the dependent claims or are explained in more detail in what follows together with the description of the preferred embodiments of the invention with reference to the drawings.

### BRIEF DESCRIPTION OF THE DRAWING

The description herein makes reference to the accompanying drawing wherein like reference numerals refer to like parts throughout the several views, and wherein:

FIG. 1 shows a schematic representation of a powder conveying pump in accordance with the invention to transport powder in a powder coating installation;

FIG. 2 shows a variation of the powder conveying pump from FIG. 1 with springs to assist the movement of the piston; and

FIG. 3 shows a further embodiment of a powder conveying pump in accordance with the invention.

### DETAILED DESCRIPTION

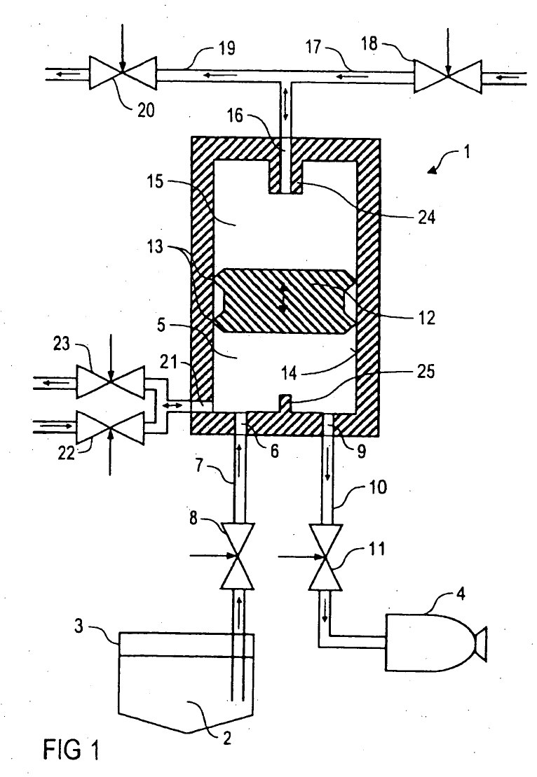

The schematic representation in FIG. 1 shows a powder conveying pump 1 that can be used in a powder coating installation to convey powder 2 serving as a coating means from a powder hopper 3 to a rotary atomizer 4 serving as the application device.

The powder hopper 3 and the rotary atomizer 4 can be of conventional construction so that in what follows a detailed description of the powder hopper 3 and the rotary atomizer 4 can be dispensed with and in this regard reference is made to the relevant technical literature.

In addition, another application device can be used in place of the rotary atomizer 4, for example, a powder spray gun. Additional parts and components, which are not shown here in the interest of simplicity, can be positioned between the powder hopper 3 and the powder conveying pump 1 and between the powder conveying pump 1 and the rotary atomizer 4.

The powder conveying pump has a working chamber 5 with a variable working chamber volume. A powder inlet 6, which is connected via a feed line 7 and a powder inlet valve 8 to the powder hopper 3, opens into the working chamber 5. With the powder inlet valve 8 open, the powder 2 can be sucked into the working chamber 5 of the powder conveying pump 1 through the feed line 7 and the powder inlet 6.

In addition, a powder outlet 9 opens into the working chamber 5 of the powder conveying pump 1. The powder outlet 9 is connected over a discharge line 10 and a powder outlet valve 11 to the rotary atomizer 4. With the powder outlet valve 11 open, the powder 2 that has been sucked into the working chamber 5 of the powder conveying pump 1 can be discharged via the powder outlet 9 and conveyed to the rotary atomizer 4.

The volume of the working chamber 5 can be changed by a piston 12 located so as to be movable in the direction of the arrow in the powder conveying pump 1. The piston preferably consists of an elastic or plastic material and has axially spaced sealing lips 13 on its outer surface, which lips 13 scrape powder remnants from an appurtenant running surface 14.

The powder conveying pump 1 further has a control chamber 15 located on the side of the piston 12 opposite the working chamber 5. A connection 16 opens into the control chamber 15 and is connected to a source of positive pressure via a positive pressure line 17 and a positive pressure valve 18. The connection 16 is further connected via a negative pressure line 19 and a negative pressure valve 20 to a source of negative pressure, which, like the source of positive pressure, is not shown in the interest of simplification. Pressure in the control chamber 15 can be adjusted through the connection 16 to control the movement of the piston 12. For the downward movement of the piston 12, the positive pressure valve 18 is opened while the negative pressure valve is closed. Pressure in the control chamber 15 rises accordingly, and the piston 12 moves down.

A further connection 21 opens into the working chamber 5 of the powder transfer pump 1 and is connected to a source of transfer air over a transfer air valve 22. With the transfer air valve 22 in the open position, transfer air is blown into the working chamber 5 of the powder transfer pump 1 through the connection 21, resulting in the powder 2 present in the working chamber 5 being discharged through the powder outlet 9 if the powder outlet valve 11 is open.

A negative pressure connection can be attached in addition to connection 21 by way of a negative pressure valve 23 to generate negative pressure in the working chamber 5 and thereby to pull the piston 12 downward. This control of the piston through negative pressure is optional, however.

Finally, the powder conveying pump 1 has a stop 24 projecting into the control chamber and a stop 25 projecting into the working chamber 5. The two stops 24, 25 limit the movement of the piston 12 and thereby set a specified maximum swept volume.

A complete operating cycle of the powder conveying pump 1 will now be described in what follows, in which the piston 12 is at the lower stop 25 at the start of the operating cycle described. The powder inlet valve 8, the powder outlet valve 11, the transfer air valve 22, the negative pressure valve 20 and the positive pressure valve 18 are closed.

At the start of the operating cycle, the powder inlet valve 8 and the negative pressure valve 20 are opened, allowing air to be sucked out of the control chamber 15. This results in a corresponding reduction in pressure in the control chamber 15 and an upward movement of the piston 12. The upward movement of the piston 12 in turn causes a reduction in pressure in the working chamber 5. As a result of this reduction in pressure, the powder 2 is sucked out of the powder hopper 3 via the powder inlet 6 into the working chamber 5.

When the piston 12 strikes the upper stop 24, the powder inlet valve 8 and the negative pressure valve 20 are closed, while the powder outlet valve 11 and the transfer air valve 22 are opened. Transfer air is blown through the connection 21 into the working chamber 5, with the result that the powder present in the working chamber 5 is discharged through the powder outlet 9.

After the powder 2 is discharged from the working chamber 5, the transfer air valve 22 is closed. The powder outlet valve 11 remains open, while the positive pressure valve 18 is opened. As a result, the piston 12 moves downward until the piston 12 finally strikes the lower stop 25, whereupon the positive pressure valve 18 is closed so that the operating cycle is completed.

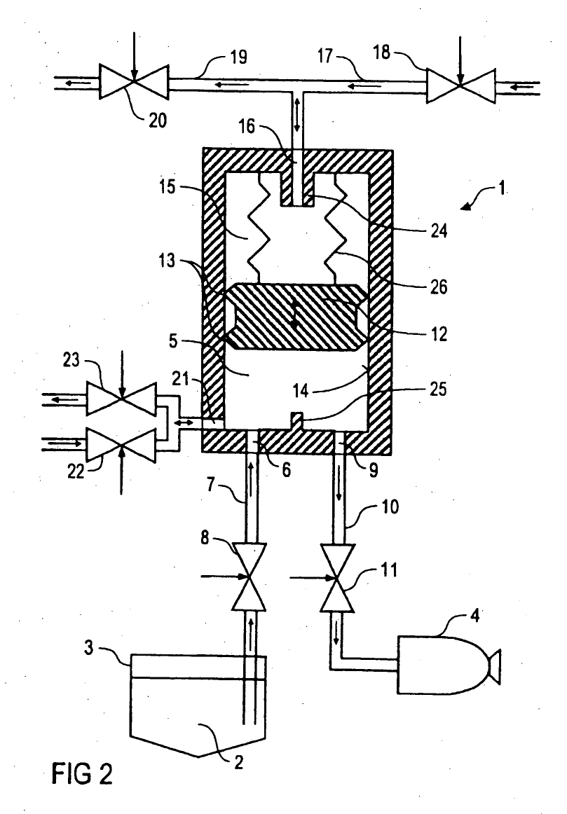

The embodiment shown in FIG. 2 is largely identical to the embodiment described previously and shown in FIG. 1 so that general reference is made to the preceding description to avoid repetitions, and identical reference numbers are used as in FIG. 2 for corresponding components.

One special feature of this embodiment is that, on the side facing the control chamber 15, the piston 12 is connected to two tension springs 26. The two tension springs 26 are attached to the housing for the powder conveying pump 1. The two tension springs 26 support the upward movement of the piston 12 when powder 2 is being sucked into the working chamber 5, and they assume the upward movement in the existence of a vacuum.

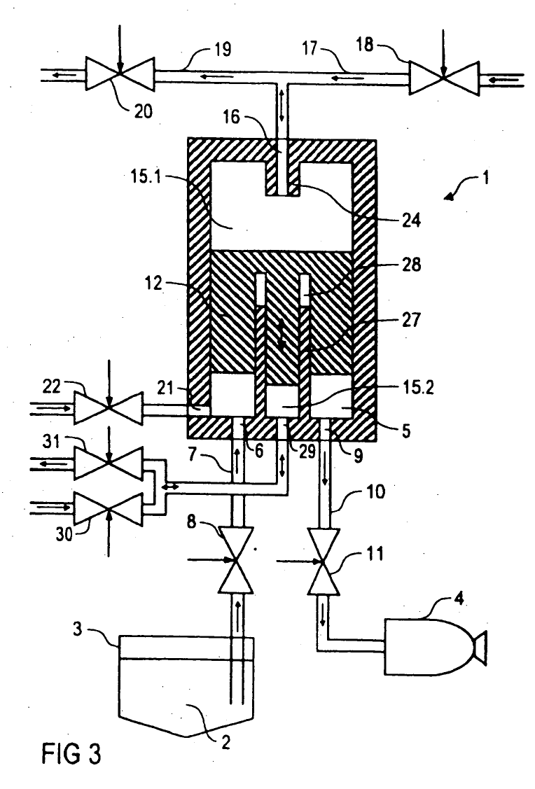

Finally, FIG. 3 shows a further embodiment which is likewise largely identical to the embodiment described previously and shown in FIG. 1, so that general reference is made largely to the preceding description to avoid repetition, and the same reference numbers are used as in FIG. 3 for corresponding components.

One special feature of this embodiment is that the powder conveying pump 1 has two control chambers 15.1, 15.2 to drive the piston 12 pneumatically, where control chamber 15.1 is located on the side of the piston 12 facing away from the working chamber 5, while control chamber 15.2 is on the same side of the piston 12 as the working chamber 5. Control chamber 15.1 can be actuated in the same way as the control chamber 15 in FIG. 1 so that reference is made to the preceding description in this regard. Control chamber 15.2 on the other hand is separated from the working chamber 5 by a canister-shaped cylindrical wall 27, with the working chamber 5 surrounding the control chamber 15.2 in annular fashion. In addition, there is an annular groove 28 in the piston 12. The wall 27 engages the annular groove 28. Alternatively, the connections 6, 29, 9 and 21 could be rearranged such that control chamber 15.2 instead surrounds the working chamber 5.

The upward movement of the piston 12 is initiated by compressed air being blown in through a connection 29 and a positive pressure valve 30 into the control chamber 15.2, which causes the piston 12 to move upward and to suck the powder 2 into the working chamber 5 through the powder inlet 6. At the downward movement of the piston 12, the positive pressure connection 30 is closed. An outlet valve 31 connected to the connection 29 is opened so that the air present in the control chamber 15.2 can be vented through the outlet valve 31 to atmosphere at the downward movement of the piston 12.

The advantage of this embodiment is that the upward movement of the piston 12 to suck the powder 2 into the working chamber 5 can take place with considerably greater force since high positive pressure can easily be generated in the control chamber 15.2, whereas the differential pressure when the piston 12 from FIG. 1 is generating suction is limited to a maximum of 1 bar.

The invention is not limited to the preferred embodiments previously described. Rather, a plurality of variants and modifications are possible that make similar use of the inventive ideas and therefore fall within its spirit and scope.

What is claimed is:

1. A powder conveying pump for a powder coating installation, comprising:
a working chamber with a variable working chamber volume;
a powder inlet opening into the working chamber to suck powder into the working chamber;
a powder outlet opening out of the working chamber to discharge the powder present in the working chamber;
a moveable piston forming one boundary surface of the working chamber and operable to suck the powder into the working chamber through the powder inlet during movement of the piston in a direction away from the working chamber; and
pneumatic means for directly driving the piston pneumatically.

2. The powder conveying pump according to claim 1 wherein the pneumatic means comprises a control chamber to drive the piston pneumatically, the piston forming one boundary surface of the control chamber.

3. The powder conveying pump according to claim 2 wherein the control chamber and the working chamber are located on opposite sides of the piston.

4. The powder conveying pump according to claim 2 wherein the control chamber and the working chamber are located on the same side of the piston.

5. The powder conveying pump according to claim 4 wherein the working chamber surrounds the control chamber in an annular fashion.

6. The powder conveying pump according to claim 4 wherein the control chamber surrounds the working chamber in an annular fashion.

7. The powder conveying pump according to claim 1 wherein the pneumatic means further comprises a first control chamber located on one face of the piston; and a second control chamber located on an opposed face of the piston; and wherein the first and the second control chambers are operable to drive the piston pneumatically in two directions.

8. The powder conveying pump according to claim 7 wherein the working chamber surrounds the control chamber in an annular fashion.

9. The powder conveying pump according to claim 7 wherein the control chamber surrounds the working chamber in an annular fashion.

10. The powder conveying pump according to claim 1 wherein the pneumatic means comprises a control chamber to drive the piston pneumatically, the piston forming one boundary surface of the control chamber, the powder conveying pump further comprising:
at least one of a positive pressure connection opening into the control chamber, the positive pressure connection operable to generate positive pressure to move the piston and to change the working chamber volume accordingly and a negative pressure connection opening into the control chamber, the negative pressure connection operable to generate negative pressure to move the piston and to change the working chamber volume accordingly.

11. The powder conveying pump according to claim 10, further comprising:
a transfer air connection opening into the working chamber, the transfer air connection operable to blow transfer air into the working chamber and discharge the powder present in the working chamber through the powder outlet.

12. The powder conveying pump according to claim 1, further comprising:
a transfer air connection opening into the working chamber, the transfer air connection operable to blow transfer air into the working chamber and discharge the powder present in the working chamber through the powder outlet.

13. The powder conveying pump according to claim 12 wherein the transfer air connection and the powder outlet are located opposite each other in the working chamber.

14. The powder conveying pump according to claim 1, further comprising:
a negative pressure connection opening into the working chamber, the negative pressure connection operable to generate a negative pressure to move the piston and to change the working chamber volume accordingly.

15. The powder conveying pump according to claim 1, further comprising:
at least one spring connected to the piston, the at least one spring operable to one of push and pull the piston.

16. The powder conveying pump according to claim 1, further comprising:
a stop located in the working chamber, the stop limiting piston movement during one of a downward movement and an upward movement.

17. The powder conveying pump according to claim 1 wherein the piston comprises at least one of an elastic material and a plastic material.

18. The powder transfer pump according to claim 1, further comprising:
a one-piece formed lip seal on an outer surface of the piston, the one-piece formed lip seal formed to scrape off powder remnants.

19. In a powder coating installation, the improvement comprising a powder conveying pump according to claim 1.

20. In a method of conveying powder in a powder coating installation, use of the powder conveying pump according to claim 1.
